(19) United States
(12) Patent Application Publication
(10) Pub. No.: US 2026/0207117 A1
(43) Pub. Date: Jul. 23, 2026

(54) NEURAL SURVIVAL MAPPING

(71) Applicant: Cochlear Limited, Macquarie University, NSW (AU)

(72) Inventors: Christopher BENNETT, Macquarie Park, NSW (AU); Helmut Christian EDER, Bemboka, NSW (AU)

(21) Appl. No.: 19/140,151

(22) PCT Filed: Jan. 17, 2024
(86) PCT No.: PCT/IB2024/050461

§ 371 Date: Jun. 17, 2025

Related U.S. Application Data
(60) Provisional application No. 63/481,227, filed on Jan. 24, 2023.

(51) Int. Cl. A61B 5/388; A61B 34/20

(52) U.S. Cl. CPC A61B 5/388; A61B 34/20; A61B 2034/2048

(57) ABSTRACT

Presented herein techniques for generating a neural survival map of neural tissue adjacent a body cavity of a recipient of an implantable medical device comprising an implantable stimulating assembly. For example, during insertion of the implantable stimulating assembly into the recipient, the implantable medical device captures a plurality of evoked responses of neural tissue adjacent to body cavity of, as well as a plurality of intra-operative measurements associated with the implantable stimulating assembly. A computing device is configured to use plurality of intra-operative measurements to determine a plurality of position estimates of the implantable stimulating assembly relative to the body cavity. The computing device uses the plurality of evoked responses and the plurality of position estimates to generate a neural survival map of the neural tissue adjacent to the body cavity.

### BACKGROUND

### Field of the Invention

Presented here are techniques for generating neural survival maps.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The first method comprises: during insertion of a stimulating assembly into a cochlea, obtaining a plurality of evoked responses from the cochlea; during insertion of the stimulating assembly into the cochlea, obtaining a plurality of position estimates of the stimulating assembly within the cochlea; and generating a neural survival map of the cochlea based on the plurality of evoked responses and the plurality of position estimates.

In another aspect, a method is provided. The method comprises: during insertion of a stimulating assembly into a cochlea, performing a plurality of intra-operative neural response measurements of the cochlea; during insertion of the stimulating assembly into the cochlea, iteratively estimating positions of the stimulating assembly within the cochlea relative to a multi-dimensional geometric model of the cochlea; and analyzing the intra-operative neural response measurements relative to the estimated positions of the stimulating assembly within the cochlea to generate a neural survival map of the cochlea.

In another aspect, a method is provided. The method comprises: obtaining a neural survival map of a cochlea, wherein a stimulating assembly is at least partially inserted into the cochlea; determining, based on the neural survival map of the cochlea, a selected placement for the stimulating assembly within the cochlea; obtaining an estimated position of the stimulating assembly within the cochlea; and determining, based on the estimated position of the stimulating assembly within the cochlea, a positional adjustment to the stimulating assembly for achieving the selected placement for the stimulating assembly within the cochlea.

In another aspect, one or more non-transitory computer readable storage media are provided. The one or more non-transitory computer readable storage media comprise instructions that, when executed by a processor, cause the processor to: obtain a plurality of evoked responses during insertion of a stimulating assembly into a body cavity of a recipient; during insertion of the stimulating assembly into the body cavity of the recipient, obtain a plurality of position estimates of the stimulating assembly within the body cavity; and generate a neural survival map of the body cavity based on the plurality of evoked responses and the plurality of position estimates.

In another aspect, a system is provided. The system comprises: a display screen; a memory storing computer readable instructions; at least one processor operable coupled to the display screen and the memory, wherein the at least one processor is configured to: obtain a plurality of intra-operative neural response measurements captured during insertion of a stimulating assembly into a body cavity, obtain a plurality of position estimates of the stimulating assembly within the body cavity captured relative to a multi-dimensional geometric model of the body cavity; and analyze the intra-operative neural response measurements relative to the estimated positions of the stimulating assembly within the body cavity to generate a neural survival map of the body cavity.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2 is a flowchart illustrating a method for implementing generation of a neural survival map according to techniques presented herein;

FIG. 3 is a graphical view of a neural survival map according to an example embodiment;

FIG. 4 is a flowchart illustrating a method for implementing determination of a positional adjustment to a stimulating assembly according to techniques presented herein;

FIG. 5 is a graphical view of a neural survival map with a representation of a positional adjustment to adjust a stimulating assembly from a current estimated placement to a selected placement, according to an example embodiment;

FIG. 6 is a schematic diagram illustrating an implantable stimulator system with which aspects of the techniques presented herein can be implemented;

FIG. 7 is a schematic diagram illustrating a vestibular stimulator system with which aspects of the techniques presented herein can be implemented; and

FIG. 8 is a flowchart illustrating a generalized method for generating a neural survival map according to techniques presented herein.

### DETAILED DESCRIPTION

Presented herein techniques for generating a neural survival map of neural tissue adjacent a body region/cavity of a recipient of an implantable medical device comprising an implantable stimulating assembly. For example, during insertion of the implantable stimulating assembly into the body cavity, the implantable medical device captures a plurality of evoked responses of neural tissue adjacent to the body cavity, as well as a plurality of intra-operative associated with the implantable stimulating assembly. A computing device is configured to use plurality of intra-operative measurements to determine a plurality of position estimates of the implantable stimulating assembly relative to the body cavity. The computing device uses the plurality of evoked responses and the plurality of position estimates to generate a neural survival map of the neural tissue adjacent to the body cavity. In some embodiments, the implantable stimulating assembly is an intra-cochlear stimulating assembly configured to be inserted into a cochlea of the recipient (e.g., the body cavity is the recipient's cochlea), and the neural survival map is a map of the recipient's surviving spiral ganglion cells (e.g., nerve cells adjacent to the cochlea).

In certain aspects, the same or different computing device uses the neural survival map to determine a selected placement (e.g., optimal position/location) for the implantable stimulating assembly with the body cavity (e.g., the recipient's cochlea). The selected placement can be used to generate a positional adjustment to the implantable stimulating assembly that, for example, aids in aligning electrodes of the implantable stimulating assembly with areas of relatively greater neural survival.

Merely for ease of description, the techniques presented herein are primarily described with reference to a specific medical device in the form of a cochlear implant system and the generation of a neural survival map of a recipient's inner ear, namely the cochlea. However, it is to be appreciated that the techniques presented herein can be implemented in/with a number of different types of medical devices to generate neural survival maps of different neural tissue regions adjacent to different body cavities of a recipient. For example, the techniques presented herein may also be partially or fully implemented by devices/systems that include hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, combinations or variations thereof, etc.

Referring specifically to the inner ear, a recipient's cochlea organ comprises a three-dimensional spiral shaped cavity within the bony labyrinth of the temporal bone. The scala tympani and scala vestibuli ducts wind around the axis of the spiral from the base to the apex and neural cells (e.g., spiral ganglion cells) distributed throughout. The cochlea is tonotopically mapped such that neural cells towards the base of the cochlea convey high frequency auditory signal and the cells towards the apex convey low frequency auditory signals. Cochleae with associated hearing loss, depending on the disease state, generally have sporadic or “patchy” distribution of neural cells. The placement of a stimulation assembly inside the cochlea may not necessarily stimulate sections of the cochlea with better concentrations of neural cells.

After surgery, an x-ray or computed tomography (CT) scan can be used to confirm the placement of a stimulating assembly within a recipient's cochlea. However, during insertion or after insertion of the stimulating assembly, a surgeon is typically unable to confirm the placement of the stimulating assembly relative to the cochlea's internal structures without intraoperative imagining such as fluoroscopy, an intraoperative x-ray, or an intraoperative CT scan. Presented herein are techniques that measure and estimate neural survival in real-time during placement of a stimulating assembly within a recipient, e.g., inside the cochlea. That is, the techniques presented herein map neural survival throughout the cochlea, and, in certain examples, determined a selected (e.g., optimal) placement of the stimulating assembly that would maximize coverage of healthy/active/responsive cells (and/or minimize coverage of unhealthy/inactive/unresponsive cells) in order to obtain the greatest possible coverage of stimulation of neural cells. Using this information, the techniques presented herein can generate an output that, for example, can recommend to the surgeon how to alter the placement of the stimulating assembly, and/or that controls a surgical robot to alter the placement of the electrode array to achieve the selected placement. Achieving the selected placement of could lead to better hearing and quality of life outcomes.

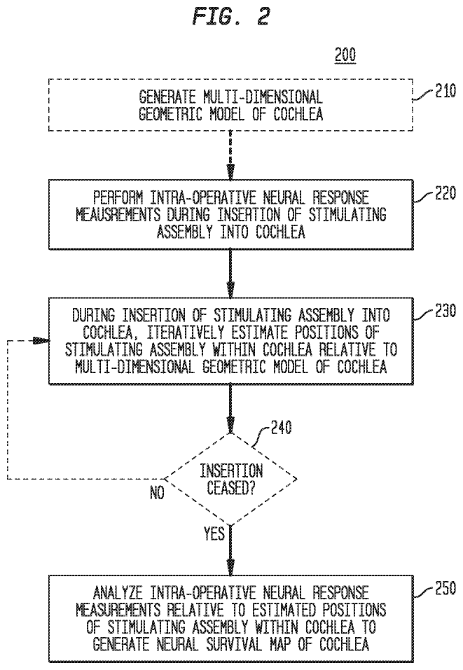

Generally, the system and methods described herein involve techniques for determining a neural survival map of the cochlea by repeatedly performing measurements while inserting a stimulating assembly into the cochlea. In some example implementations, the determined neural survival map can be used to optimize the position of the stimulating assembly in the cochlea. As described further below with reference to FIGS. 2, 3, 4, 5, and 8, the system and methods described herein are comprised a number of different functional components/sub-systems. These functional components/sub-system can include, for example: (1) a sub-system to capture intra-operative measurements of neural responses in real-time during insertion of an stimulating assembly into a cochlea; (2) a sub-system to estimate the location of the stimulating assembly relative to a multi-dimensional geometric cochlea model; (3) a sub-system to generate a neural survival map; and (4) a sub-system to calculate the selected placement of the stimulating assembly and/or to calculate a positional adjustment to the stimulating assembly to achieve the selected placement.

### Example System

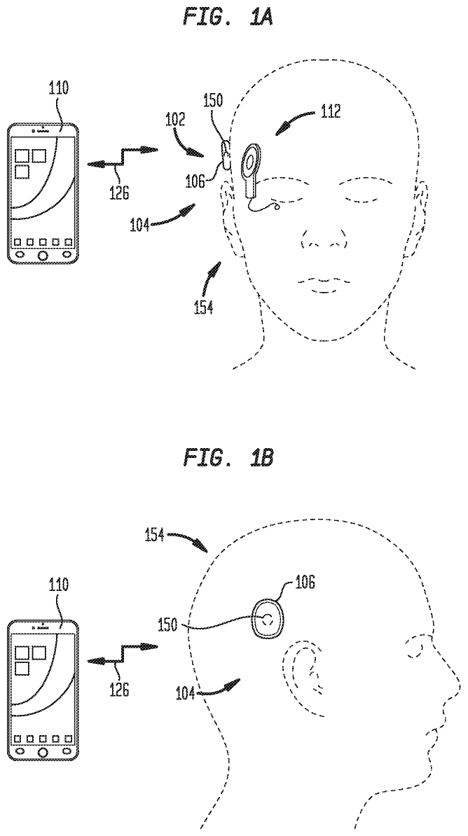

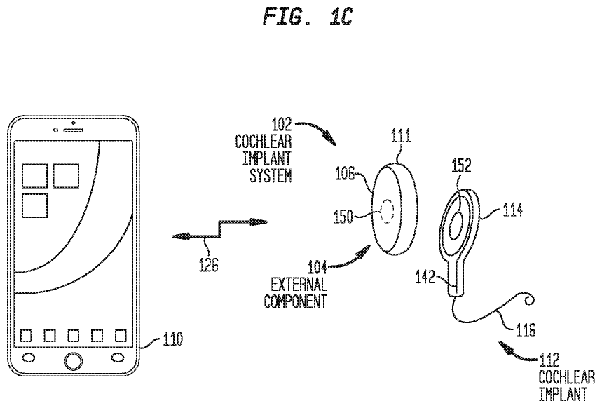

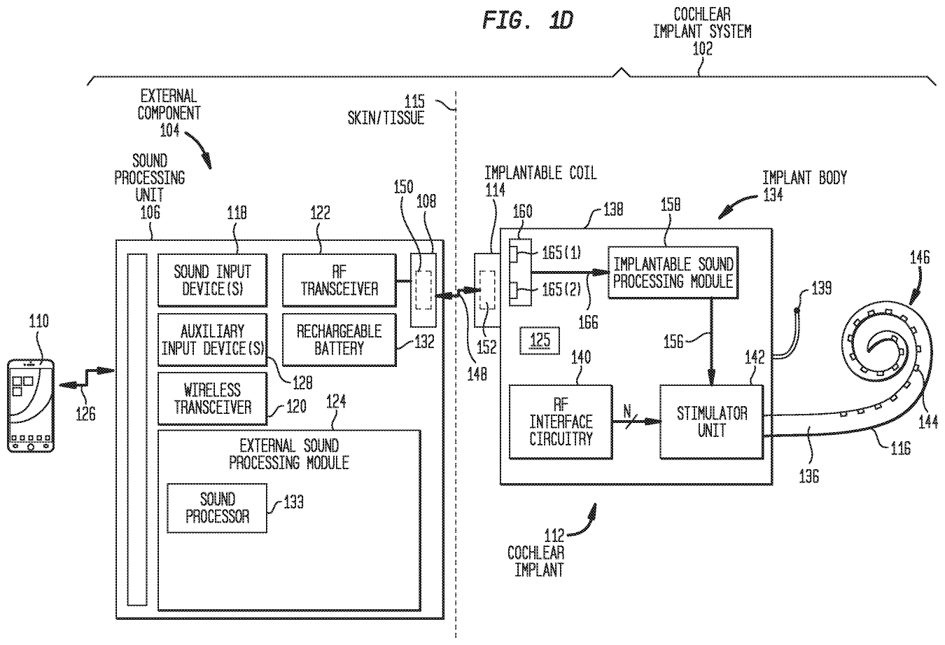

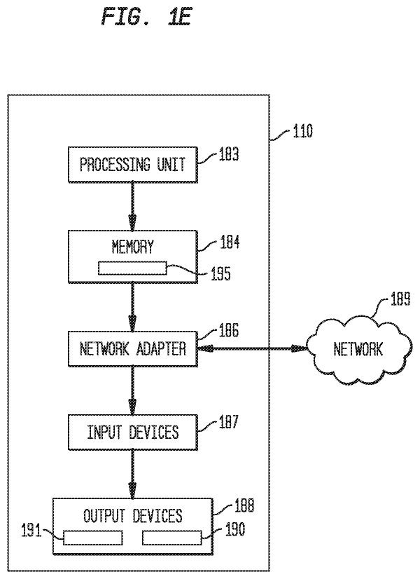

FIGS. 1A-1D illustrates an example cochlear implant system 102 with which aspects of the techniques presented herein can be implemented. The cochlear implant system 102 comprises an external component 104 that is configured to be directly or indirectly attached to the body of the user, and an internal/implantable component 112 that is configured to be implanted in or worn on the head of the user. In the examples of FIGS. 1A-1D, the implantable component 112 is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

In the examples of FIGS. 1A-1D, the external component 104 comprises a sound processing unit 106, an external coil 108, and generally, a magnet fixed relative to the external coil 108. The cochlear implant 112 includes an implantable coil 114, an implant body 134, and an elongate stimulating assembly 116 configured to be implanted in the user's cochlea. In one example, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head 154 (e.g., includes an integrated external magnet 150 configured to be magnetically coupled to an internal/implantable magnet 152 in the implantable component 112). The OTE sound processing unit 106 also includes an integrated external (headpiece) coil 108 (the external coil 108) that is configured to be inductively coupled to the implantable coil 114.

It is to be appreciated that the OTE sound processing unit 106 is merely illustrative of the external devices that could operate with implantable component 112. For example, in alternative examples, the external component 104 may comprise a behind-the-ear (BTE) sound processing unit configured to be attached to, and worn adjacent to, the recipient's ear. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

Although the cochlear implant system 102 includes the sound processing unit 106 and the cochlear implant 112, as described below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. 1E, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on or near the sound processing unit 106. However, it is to be appreciated that one or more input devices may include additional types of input devices and/or less input devices (e.g., the short-range wireless transceiver 120 and/or one or more auxiliary input devices 128 could be omitted).

The sound processing unit 106 also comprises the external coil 108, a charging coil 130, a closely-coupled radio frequency transmitter/receiver (RF transceiver) 122, at least one rechargeable battery 132, and an external sound processing module 124. The external sound processing module 124 can be configured to perform a number of operations which are represented in FIG. 1D by a sound processor 133. The sound processor 133 can be formed by one or more processors (e.g., one or more Digital Signal Processors (DSPs), one or more uC cores, etc.), firmware, software, etc. arranged to perform operations described herein. That is, the sound processor 133 can be implemented as firmware elements, partially or fully implemented with digital logic gates in one or more application-specific integrated circuits (ASICs), partially or fully in software, etc. Although FIG. 1D illustrates a sound processor 133 as being implemented/performed at the external sound processing module 124, it is to be appreciated this element (e.g., functional operations) could also or alternatively be implemented/performed as part of the implantable sound processing module 158, as part of the external device 110, etc.

In the example of FIGS. 1A-1D, the implantable component 112 comprises an implant body (main module) 134, a lead region 136, and the intra-cochlear stimulating assembly 116, all configured to be implanted under the skin (tissue) 115 of the user. The implant body 134 generally comprises a hermetically-sealed housing 138 that includes, in certain examples, at least one power source 125 (e.g., one or more batteries, one or more capacitors, etc.) 125, in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the internal/implantable magnet 152 is fixed relative to the implantable coil 114. The external magnet 150 and the internal/implantable magnet 152 fixed relative to the external coil 108 and the internal/implantable coil 114, respectively, facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient or user (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals (stimulation signals) that represent electrical stimulation for delivery to the recipient.

As noted, FIG. 1D illustrates an embodiment in which the external sound processing module 124 in the sound processing unit 106 generates the output control signals. In an alternative embodiment, the sound processing unit 106 can send less processed information (e.g., audio data) to the implantable component 112 and the sound processing operations (e.g., conversion of input sounds to output control signals 156) can be performed by a processor within the implantable component 112.

In FIG. 1D, according to an example embodiment, output control signals (stimulation signals) are provided to the RF transceiver 122, which transcutaneously transfers the output control signals (e.g., in an encoded manner) to the implantable component 112 via external coil 108 and implantable coil 114. That is, the output control signals (stimulation signals) are received at the RF interface circuitry 140 via implantable coil 114 and provided to the stimulator unit 142. The stimulator unit 142 is configured to utilize the output control signals to generate electrical stimulation signals (e.g., current signals) for delivery to the user's cochlea via one or more of the stimulating contacts (electrodes) 144. In this way, cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, an example embodiment of the cochlear implant 112 can include a plurality of implantable sound sensors 165(1), 165(2) that collectively form a sensor array 160, and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient or user (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 165(1), 165(2) of sensor array 160 in generating stimulation signals for delivery to the user.

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include random access memory (RAM), read only memory (ROM), EEPROM (Electronically-Erasable Programmable Read-Only Memory), flash memory, optical disc storage, magnetic storage, solid state storage, or any other memory media usable to store information for later access. By way of example, and not limitation, the memory 184 can include wired media such as a wired network or direct-wired connection, and wireless media such as acoustic, RF, infrared and other wireless media or combinations thereof. In certain embodiments, the memory 184 comprises neural survival analysis logic 195 that, when executed, enables the processing unit 183 to perform aspects of the techniques presented.

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include, a display 190 (e.g., a liquid crystal display (LCD)) and one or more speakers 191, among other output devices for presentation of visual or audible information to the recipient, a clinician, an audiologist, or other user.

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

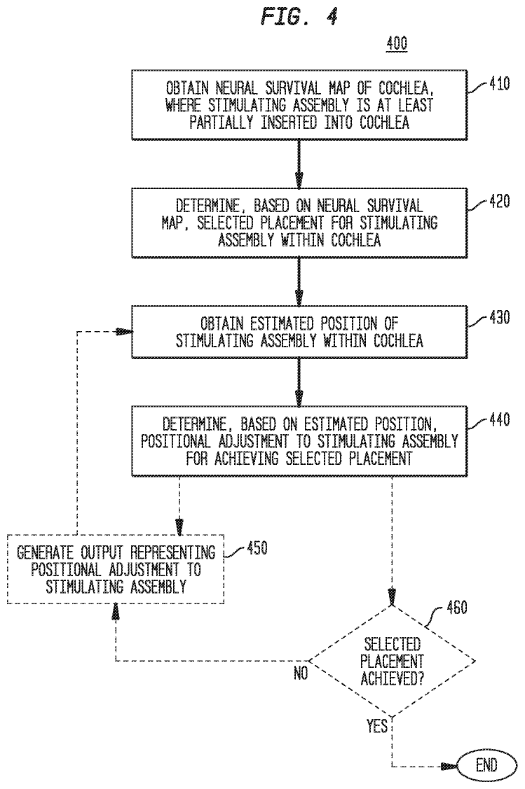

As noted, presented herein are techniques for the determination/generation and use of neural survival maps from, as explained in detail herein, objective measurements obtained/captured via components of an implantable stimulation assembly, such as stimulation assembly 116 of FIG. 1D. In certain embodiments, these objective measurements are combined with physical measurements of electrode placement to determine the neural health of the nerves stimulated by the stimulation assembly, as described further below with reference to FIGS. 2, 3, and 8. In certain embodiments, a neural survival map can be used to determine a selected placement of the stimulation assembly (and individual electrodes thereof), and determine a positional adjustment to the stimulation assembly for achieving the selected placement based on the current estimated placement of the stimulation assembly, as described further below with reference to FIGS. 4, 5, and 8.

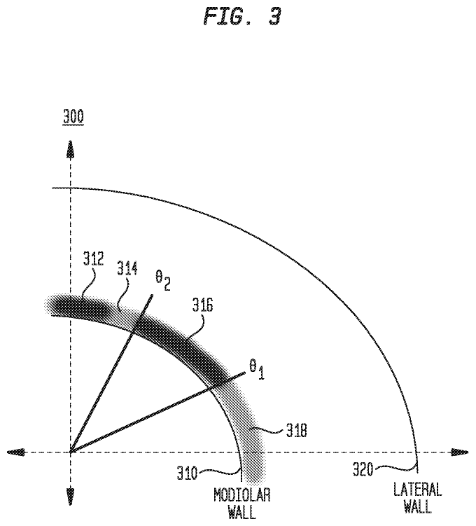

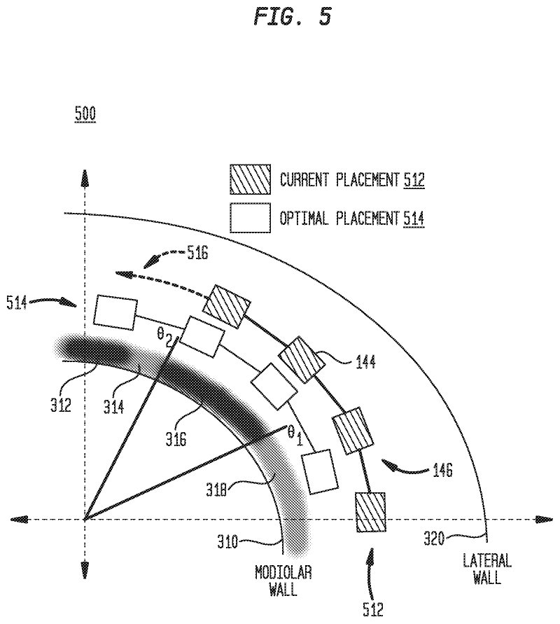

Next, example techniques for generating a multi-dimensional geometric model of a cochlea and for estimating placement (position/location) of a stimulating assembly within the cochlea are described below, and then various uses for these techniques will be described with reference to the programmatic flowcharts of FIGS. 2, 4, and 8 (and also referring to the graphical views shown in FIGS. 3 and 5).

### Generation of a Multi-Dimensional Geometric Cochlea Model

In general, the purpose of a multidimensional geometric model is to create a physical representation of the cochlea (or other area of the recipient's body) for the purpose of creating a locational reference for the varying placements of a stimulation assembly (electrode array) and regions of low neural survival or high neural survival.

As noted above, the cochlea is a three-dimensional spiral structure within the bony labyrinth. The scala tympani, scala vestibuli and scala media twist around the central axis of the cochlea (modiolus, mid-modiolar axis) from the base to the apex (helicotrema). The modiolus contains the cochlea nerve. The description of the location of points in the cochlea is in polar coordinates, where the mid-modiolar axis is the origin. At the base of the cochlea, on the scala tympani, is the round window. The chord from the round, passing through the round window to the lateral wall, and the orthogonal chord form the x-axis and γ-axis, respectively. The angular coordinate's origin is the vector from the mid-modiolar axis to the round window and the radial distance is measured from the mid-modiolar axis.

The dimensions (length, width, and height) of the cochlea can be measured from preoperative medical imaging. The cochlea dimensions are input to algorithms that estimate the geometry of the ducts the cochlea (i.e., scala tympani, scala vestibuli and scala media). In particular, a first algorithm uses: (A) a hyperbolic spiral to estimate the modiolar wall (Equations 1 and 2, below); (B) a hyperbolic spiral to estimate the lateral wall (Equations 1 and 3, below); and (C) an ellipse to estimate the cochlear duct (Equations 4 and 5, below) spanning from the modiolar wall to the lateral wall.

$$\begin{matrix}Equation 1 & \\ {\hat{z}}_{θ} = f ( θ ) = {β}_{0} + \frac{{β}_{1} θ + {β}_{2} {e}^{{β}_{3} θ}}{{B}_{3} height} + ε , while 0 \leq \hat{z} \leq height & ( 1 )\end{matrix}$$

where \hat{z}_{θ} is the estimation of the average height of the modiolar wall spiral or lateral wall spiral at a given angular deviation θ, height is the measured height of the cochlear, βᵢ is the i^{th} coefficient term of the model and ε is the error term.

$$\begin{matrix}Equation 2 & \\ & ( 2 )\end{matrix}$$

$${\hat{r}}_{modilous , θ} = f ( θ , {z}_{θ} , A , B ) = {β}_{0} + \sqrt{{β}_{1} \frac{{β}_{2} \times A \times cos ( θ )}{{β}_{3} θ} + \frac{{β}_{4} \times B \times cos ( θ )}{{β}_{5} θ}} + {β}_{6} z + ε$$

where \hat{r}_{modilous,θ} is the distance from the mid-modiolar axis of the modiolar wall at a at a given angular deviation θ, z_{θ} is the average height of the modiolar wall spiral or lateral wall spiral at θ, A is the length of the cochlea, B is the width of the cochlea, βᵢ is the i^{th} coefficient term of the model and ε is the error term.

$$\begin{matrix}Equation 3 & \\ & ( 3 )\end{matrix}$$

$${\hat{r}}_{lateral , θ} = f ( θ , {z}_{θ} , A , B ) = {β}_{0} + \sqrt{{β}_{1} \frac{{β}_{2} \times A \times cos ( θ )}{{β}_{3} θ} + \frac{{β}_{4} \times B \times cos ( θ )}{{β}_{5} θ}} + {β}_{6} z + ε$$

where \hat{r}_{lateral,θ} is the distance from the mid-modiolar axis of the modiolar wall at a at a given angular deviation θ, z_{θ} is the average height of the modiolar wall spiral or lateral wall spiral at θ, A is the length of the cochlea, B is the width of the cochlea, βᵢ is the i^{th} coefficient term of the model and ε is the error term.

$$\begin{matrix}Equation 4 & \\ & ( 4 )\end{matrix}$$

$$a = {r}_{lateral} - {r}_{modiolus} = {ellipse}_{θ , φ} = f ( φ , a ) = {β}_{0} + \frac{{β}_{1} a}{\sqrt{{( {β}_{2} \times a \times cos ( φ ) )}^{2} + {( {β}_{3} \times a \times sin ( φ ) )}^{2}}} + ε$$

where a is the length of the duct, ellipsee_{θ,φ} is the distance from the centre of the duct for a given angular deviation θ of the cochlea, φ is the angle along the ellipse, βᵢ is the i^{th} coefficient term of the model and ε is the error term.

The coefficients of the models can be estimated by an algorithm minimizing the difference between the model outputs and corresponding measured points of many (hundreds to thousands) medically imaged cochlea (both in vivo and ex vivo). The many imaged cochleae are labelled (measurements of the position of the structures of the cochlea) using imaging software with measurement tools and/or automated processing software. Algorithms used for estimating coefficients can include recursive least squares and/or optimisation (e.g., Nelder-Mead, Newton-Conjugate-Gradient).

Equations 1 through 4 are used to estimate points from the base of the cochlea to the apex, in terms of height from the abase and angular deviation, that substantiate the duct. Voxels are created from the positions of sets of points in close proximity to each other. The voxels are ascribed with physical and/or mechanical properties to govern the placement of the stimulating assembly within. For example, the physical properties of the cochlea are such that the stimulating assembly physically cannot pass through the modiolar wall and/or lateral wall.

### Estimating Position/Location of Stimulating Assembly

In some example embodiments, the placement (position and/or location) of a stimulation assembly inside of the cochlea during insertion is estimated based on one or more intra-operative 1 measurements, such as two-point impedance measurements, four-point impedance measurements, transimpedance measurements, etc. In some embodiments, the intra-operative measurements can be combined with accelerometer measurements and/or other sensor measurements.

For example, as the stimulation assembly is being inserted into the cochlea, the electrodes of the electrode array will sequentially contact the fluid of the cochlea (perilymph), which in turn forms an electrical circuit where the measured impedances will be indicative of a closed circuit. That is, during insertion, when an electrode is inserted into the cochlea (and contacts the fluid), an open circuit is no longer registered and, given the known physical dimensions of the stimulation assembly, it is possible to determine the length of the stimulating assembly located inside of the cochlea relative to the insertion point (e.g., round window or cochleostomy). For example, if electrodes 18-22 do not register an open circuit and electrodes 1-17 register an open circuit, it is discerned that length of the array from electrode 18 and onwards is inside of the cochlea.

In addition, when the stimulating assembly is fully inserted into the cochlea, features of the current transimpedance measurements and historical transimpedance measurements can be input to a probabilistic models (generalized by Equation 5 below) to estimate positional features of the stimulation assembly such as depth of insertion of electrodes, proximity to the modiolar wall and angle. The value of the placement feature of highest probability is selected to be the placement feature. The probabilistic model can be in the form of Naïve Bayes, Hidden Markov Mode, Bayesian Network, etc.

$$\begin{matrix}Equation 5 & \\ & ( 5 )\end{matrix}$$

$$P ( {placement}_{feature} ) = f ( {transimpedance}_{features , t} , … , {transimpedance}_{feature , t - n} ) + 𝒩$$

where P(placement_{feature}) is the probability of the placement feature occurring given current trans-impedance features transimpedance_{features,t} and historical transimpedance_{features,t-i}, t−i is a previous timestep, t−n is the maximal previous timestep and [symbol] is the probabilistic uncertainty term.

At the given time t, the placement features are used as anchor points to locate the stimulation assembly inside of the cochlea with each electrode being given a location in polar space. This allows for any measures taken at specific electrodes to be associated with the same location in the cochlea. Multiples of the same type of intraoperative measurement, associated at the same point in space, can be aggregated to increase measurement precision.

Specific transimpedance features are a product of the physical anomalies of the recipient's cochlea at certain locations. When different electrodes pass by these locations, they will record similar transimpedance feature values. As the stimulation assembly is being inserted, locations inside the cochlea have measurements associated with them. Each electrode has its measurements (as a time series) discretized and each discretized portion is compared to measurements located throughout the cochlea. If multiple electrodes register higher correlations to specific locations, knowing the physical properties of the stimulation assembly (i.e., spacing between electrodes), the distance that the stimulation assembly has travelled can be estimated. Distance travelled estimates can be combined with other placement feature estimates to improve precision and accuracy.

In certain examples, an accelerometer can be attached to/incorporated in a medical apparatus used to insert the stimulation assembly, or an accelerometer probe can be attached to the lead of the stimulation assembly to capture accelerometer data indication of movement of the stimulation assembly. During insertion, the captured accelerometer data indicates one of forward movement into the cochlea, no movement, or reverse movement out from the cochlea. This movement data can be used to correct estimates of changes in electrode position in circumstances where changes in the location of the stimulation assembly are not congruent, for example.

In certain embodiments, when the impedance or transimpedance data recorded over a specific period registers a threshold of minimal change, the system registers that the insertion has halted. Alternatively, when the accelerometer data indicates no movement of the period, the system registers that the insertion has halted. This detection that the insertion of the stimulation assembly has ceased triggers the system (e.g., via software, logic, computer-readable instructions, etc.) to generate the neural survival map.

### Process Flow for Generating Neural Survival Map

FIG. 2 is a programmatic flowchart illustrating an example method 200 for generating a neural survival map, in accordance with certain embodiments presented. As shown in FIG. 2, after method 200 commences, the system generates a multi-dimensional geometric model of a cochlea at operation 210. In some example embodiments, operation 210 may include retrieving user input data indicating the dimensions (e.g., length, width, height) of the cochlea, and generating the multi-dimensional geometric model approximating the cochlea based on the user input data. In some other example embodiments, operation 210 may include retrieving pre-operative medical imaging scan data (e.g., CT, MRI, etc.), which is processed by an algorithm to generate voxels capturing the multi-dimensional structure of the cochlea as described above.

In some example embodiments, the flow of method 200 can optionally include receiving a manual input from a user (e.g., the surgeon or other medical professional) to register that the insertion of the stimulating assembly into the cochlea of the patient has started, which triggers operation 220. Alternatively, operation 220 could be triggered automatically based on captured data.

At operation 220, during insertion of the stimulating assembly into the cochlea, the system performs a plurality of intra-operative neural response measurements at a high temporal frequency during insertion of the stimulating assembly into the cochlea. For example, while the insertion is being conducted, the cochlear implant system executes an alternating regime of intra-operative measurements comprising impedance measurements and electrically evoked compound action potential (ECAP) measurements from stimulation. The ECAP measurements are processed to form neural response telemetry (NRT) measurements. Thus, operation 220 may include performing impedance measurements, performing ECAP measurements, generating NRT measurements from ECAP measurements, or a combination thereof. These neural response measurements are stored in a memory device as they are recorded. Storage of the neural response measurements in the memory can help to reduce or eliminate the need for using trial-and-error techniques, for example. In some example embodiments, operation 220 may also include mapping an internal structure of the cochlea while performing the intra-operative neural response measurements.

Also, during insertion of the stimulating assembly into the cochlea, at operation 230, the system iteratively estimates a real-time position of the stimulating assembly within the cochlea relative to the multi-dimensional geometric model of the cochlea. As the insertion is being conducted and as the measurements are being made, models that capture the relationships between these measurements and physical location features are used to estimate the placement (e.g., position and/or location) of the stimulating assembly inside the cochlea in relation to the cochlea model. The estimation of the physical placement (position, location) of the stimulating assembly in the cochlea is then used to register (or “collocate”) the intra-operative neural response measurements (e.g., ECAP, NRT) to the corresponding position/location of the stimulating assembly (and/or individual electrodes thereof) within the cochlea. In some example embodiments, operation 230 can include performing impedance measurements, performing transimpedance measurements, performing accelerometer measurements, or a combination thereof.

In some example embodiments, the system can optionally determine whether the stimulating assembly is still being inserted into the cochlea or insertion has ceased at operation 240. While the stimulating assembly is still being inserted (No at operation 240), operations 220 and 230 are repeated iteratively. When the system determines that insertion has ceased (Yes at operation 240), such as by detecting a minimal change in the values of the intra-operative neural response measurements as described above, the flow of method 200 may then proceed to operation 250. In some other example embodiments, operation 240 may not be performed, in which case the flow of method 200 proceeds to operation 250 directly from operation 230.

In operation 250, the system analyzes the intra-operative neural response measurements relative to the estimated positions of the stimulating assembly within the cochlea to generate a neural survival map of the cochlea. For example, the neural measurements that have been registered (collocated) to various positions of the cochlea model are processed to form the neural survival map.

Throughout the course of the insertion, the location of the stimulating assembly has periodically/continuously been estimated with electrodes of the electrode array of the stimulating assembly being given associated polar coordinates. When intra-operative measurements are made during insertion, they are associated to the estimated position of the electrode at the particular point in time. Measures of neural activity (e.g., ECAP, NRT, etc.) are measured throughout the insertion and are associated with locations in the cochlea. With a standard set of stimulation levels, a neural response benchmark is created. With a set stimulation level, sections of the cochlea with greater remaining neural tissue will provoke a greater measured response, while sections of the cochlea with lesser neural tissue will provoke a smaller measured response. The measures of neural activity are normalized.

Thus, the system is configured to provide stimulation at a known magnitude of current, and measure neural responses, where a greater magnitude of neural response indicates higher neural survival, while a lesser magnitude of neural response indicates lower neural survival. By estimating the physical position of the stimulating assembly, the system can collocate these neural response measurements to the physical locations in a map (e.g., a 2D map or a 3D map).

As described herein, a neural survival map (refer to example shown in FIG. 3) highlights regions of high neural activity and low neural activity, for example. In some example embodiments, the generation of the neural survival map may allow for adjustment of the position of the stimulating assembly after an initial placement thereof, as described further below with reference to FIGS. 4 and 5.

FIG. 3 illustrates an example neural survival map (or neural activity map) according to an example embodiment. The neural survival map 300 of FIG. 3 maps the neural health of the cochlea, and may be generated at operation 250 of FIG. 2, for example. The light shaded regions 314 and 318 adjacent to the modiolar wall 310 have measures indicative of low neural survival. The dark shaded regions 312 and 316 adjacent to the modiolar wall 310 have measurements indicative of high neural survival. In other words, the regions 312 and 316 are indicative of good neural health in those regions, while the regions 314 and 318 are indicative of poor neural health of neuron death within those regions, according to the mapping techniques described herein. One specific region of high neural survival (region 316) was registered between angular deviation θ₁ and θ₂.

In some example embodiments, the system may utilize one or more neural activity thresholds for distinguishing active regions (e.g., healthy/responsive/live regions 312, 316) of the cochlea to target for alignment with electrodes of the stimulating assembly from inactive regions (e.g., unhealthy/unresponsive/dead regions 314, 318) of the cochlea to avoid alignment with electrodes of the stimulating assembly. In some example embodiments, the system is configured to determine a selected placement for the stimulating assembly within the cochlea that maximizes coverage of active regions having high neural survival (e.g., healthy/responsive/live regions 312, 316) and/or minimizes coverage of inactive regions having low neural survival (e.g., unhealthy/unresponsive/dead regions 314, 318).

### Process Flow for Determining Positional Adjustment to Achieve Selected Placement

FIG. 4 is a programmatic flow chart illustrating an example method 400 for determining a positional adjustment to achieve a selected placement of a stimulating assembly within a cochlea, in accordance with certain embodiments presented herein. As shown in FIG. 4, the system obtains a neural survival map of the cochlea at operation 410, where the stimulating assembly is at least partially inserted into the cochlea. The neural survival map can be obtained (e.g., retrieved) from memory or can be obtained using, for example, the flow of method 200 shown in FIG. 2, such as by collocating intra-operative neural response measurements to estimated positions/locations of the stimulating assembly within the cochlea, for example.

Once the neural survival map is obtained at operation 410, the system determines, based on the neural survival map, a selected placement for the stimulating assembly within the cochlea at operation 420. For example, the selected placement may correspond to a selected placement of the stimulating assembly (and/or the position/location of its individual electrodes) that is calculated at operation 420 by maximizing a coverage statistic through an optimization algorithm (e.g., to maximize electrode alignment with and coverage of active regions (healthy/responsive/live regions) and/or minimize electrode alignment with and coverage of inactive regions (unhealthy/unresponsive/dead regions)).

In certain embodiments, the selected placement (the optimal position/location) for the stimulating assembly determined at operation 420 of FIG. 4 is the maximal collocation of the electrodes with the regions of high or higher neural activity. For example, each electrode can have an associated collocation metric where the value of the collocation metric is the magnitude of neural activity associated with its current location in the cochlea. In certain embodiments, the system attempts to maximize the summated collocation metric by shifting the stimulating assembly to hypothetical locations in the cochlea, retrieving the collocation metric per electrode, and summating the collocation metrics.

As noted, the frequency allocation of the cochlea is tonotopically mapped. In certain examples, additional value or weight is given to a hypothetical stimulating assembly location's summated collocation metric based on the degree of coverage of a plurality of frequencies. Thus, the selected placement (the calculated optimal position/location) of the stimulating assembly corresponds to the hypothetical location/position of the stimulating assembly with the maximum summated collocation metrics.

In some example embodiments, operation 420 includes determining a placement for the stimulating assembly that maximizes alignment of electrodes of the stimulating assembly with populations of surviving nerve cells based on the neural survival map. In some example embodiments, operation 420 includes identifying one or more “active regions” of the cochlea (regions of relatively high neural survival) having an amount of neural response activity above a threshold based on one or more of the intra-operative neural response measurements, and selecting a placement for the stimulating assembly that targets alignment of one or more electrodes of the stimulating assembly with the one or more active regions of the cochlea. In some example embodiments, operation 420 includes identifying one or more “inactive regions” (regions of low neural survival) of the cochlea having an amount of neural response activity above a threshold based on one or more of the intra-operative neural response measurements, and selecting a placement for the stimulating assembly that avoids alignment of one or more electrodes of the stimulating assembly with the one or more inactive regions of the cochlea. In certain example embodiments, operation 420 can optionally include filtering possible placements for the stimulating assembly within the cochlea according to a constraint to exclude positions that are not physically achievable with a selected type of electrode of the stimulating assembly based on the multi-dimensional geometric model of the cochlea.

At operation 430, the system obtains an estimated position of the stimulating assembly within the cochlea. In some example embodiments, operation 430 includes estimating a current position of the stimulating assembly within the cochlea relative to a multi-dimensional geometric model of the cochlea. In some example embodiments, operation 430 includes capturing one or more measurements (e.g., impedance measurements, transimpedance measurements, accelerometer measurements, or a combination thereof), and estimating a current position of the stimulating assembly within the cochlea based on the one or more measurements.

At operation 440, the system determines, based on the estimated position of the stimulating assembly within the cochlea, a positional adjustment to the stimulating assembly within the cochlea for achieving the selected placement. For example, the system may determine the difference in position/location between the current placement of the stimulating assembly and the selected placement (the optimal position/location) of the stimulating assembly.

In some example embodiments, operation 440 may include comparing the estimated position of the stimulating assembly within the cochlea with the selected placement for the stimulating assembly, and determining a direction (e.g., inward/distally/apically vs. outward/proximally/basally) and a magnitude (amount, distance, length, angular insertion depth, etc.) of the positional adjustment based on the comparing.

In some example embodiments, at operation 450, the system can generate an output representing the positional adjustment to the stimulating assembly for achieving the selected placement (optimal position/location) for the stimulating assembly. In some examples (e.g., in the case of manual operation by a surgeon), operation 450 may include generating an output to display the neural survival map and a representation of the positional adjustment to the stimulating assembly for achieving the selected placement (the optimal position/location) for the stimulating assembly within the cochlea on a display device (e.g., for viewing by the surgeon). In other examples (e.g., in the case of automated operation by a robotic surgical device), operation 450 may include generating an output to control a robotic surgical device according to adjust positioning of the stimulating assembly within the cochlea based on the positional adjustment to the stimulating assembly. After the output is generated at operation 450 (e.g., displayed on the display device, or transmitted to the surgical robot), the flow of method 400 may loop back to repeat operation 430 and operation 440 (e.g., to update the calculations after a corresponding positional adjustment of the stimulating assembly has been made by the surgeon or the surgical robot).

In certain embodiments, the system may determine and provide a recommendation to alter insertion of the stimulating assembly at operations 440 and 450. On several key electrodes, the difference in electrode positions/locations between the current placement (the current estimated position/location) and the selected placement (the calculated optimal location/position) in terms of the polar coordinates (primarily angular deviation) is the degree that the stimulating assembly should be altered by the surgeon or surgical robot. This positional adjustment value is calculated (at operation 440 of FIG. 4), and may then be displayed to the surgeon or transmitted to the surgical robot (at operation 450 of FIG. 4), for example. As mentioned above, the positional adjustment may have a direction component in addition to a magnitude component.

In some example embodiments, at operation 460, the system can optionally determine whether the selected placement (the optimal position/location) for the stimulating assembly has been achieved. If the selected placement has not yet been achieved (No at operation 460), then the system can generate an output representing the positional adjustment to the stimulating assembly at operation 450, and repeat operation 430 and operation 440. If the selected placement of the stimulating assembly has been achieved (Yes at operation 460), then the flow of method 400 of FIG. 4 ends. However, in some other example embodiments, operation 460 may not be performed.

In certain embodiments, the system can validate or confirm selected placement of the stimulating assembly at operations 440 and 460. Once a new placement (location/position) of the stimulating assembly has occurred (e.g., after displaying the neural survival map and the calculated positional adjustment to the stimulating assembly at operation 450 of FIG. 4), the system recalculates the current estimated position of the stimulating assembly (repeats operation 430 of FIG. 4), and again compares this with the selected placement (the optimal position/location) of the stimulating assembly (repeats operation 440 of FIG. 4). Thus, if the current placement (the estimated current position/location) of the stimulating assembly differs from the selected placement (the calculated optimal position/location) of the stimulating assembly (No at operation 460 of FIG. 4), the system recalculates the amount that the stimulation assembly should be altered (repeats operation 440 of FIG. 4), and presents the recalculated amount to the user (repeats operation 450 of FIG. 4). If the current placement of the electrode array is equivalent to the selected placement of the stimulating assembly (Yes at operation 460 of FIG. 4), then the flow of method 400 of FIG. 4 ends.

Thus, the system is configured to do a final estimation of placement of the stimulating assembly inside the cochlea at the end of insertion, and determine whether or not the current placement best collocates to areas of higher neural survival. If not collocated to areas of high neural survival, then the system is configured to estimate a degree of manipulation or adjustment of the stimulating assembly to move it into a better position in order to improve collocation of the electrodes of the stimulating assembly with areas of higher neural survival.

As described herein, the neural survival map (which highlights regions of high neural activity and low neural activity) and the representation of the positional adjustment to the stimulating assembly (refer to example shown in FIG. 5) may allow for optimized adjustment of the position/location of the stimulating assembly within the cochlea after an initial placement thereof at least partially within the cochlea.

FIG. 5 illustrates a neural survival map (or neural activity map) and a representation of a positional adjustment to the stimulating assembly, according to an example embodiment. The neural survival map 500 of FIG. 5 may be displayed on a display device at operation 450 of FIG. 4, for example. The neural survival map 500 indicates a positional adjustment 516 (e.g., determined at operation 440 of FIG. 4) that is to occur with respect to the current placement 512 of the stimulating assembly (e.g., obtained at operation 430 of FIG. 4) in order to achieve the selected placement 514 (e.g., determined at operation 420 of FIG. 4) for the stimulating assembly, so as to achieve the best collocation (or at least an improved collocation) of the electrodes 144 (forming electrode array 146) of the stimulating assembly with the active regions of higher neural survival (dark grey shaded areas) (e.g., healthy/responsive/live region 316). In this circumstance, the positional adjustment 516 indicates that the stimulation assembly is to be inserted further into the cochlear (direction component) by the difference in angular insertion depth (magnitude component).

Thus, the system described herein is configured to produce a graphical user interface (GUI) element that displays an image of the cochlea, distinguishes areas of higher neural survival from areas of lower neural survival, and indicates a current placement (current position/location) of the stimulating assembly in relation to a selected placement (optimal position/location) of the stimulating assembly. Further, the system may provide guidance on how to manipulate or adjust the stimulating assembly in order to actually achieve the best or ideal placement (e.g., insert 1 mm farther, pull back 1 mm) within the cochlea. The system can iteratively re-run measurements and repeat the calculations in a loop until the system detects that the selected placement is achieved.

### Application to Robotic Assisted Surgery

In some example embodiments, the system and techniques described herein can be applied to implement robotic assisted surgery. Robotic assisted surgery involves the insertion of the stimulating assembly into the cochlear through motive action provided by an electronically controlled actuator. As the actuator is electronically controlled, the length of the stimulating assembly inside the cochlea is known with high precision. This enables an error of the locational estimate of the position of the stimulating assembly throughout surgery to be minimized, and the accuracy of the Monte Carlo stimulating assembly placement algorithm and subsequent neural survival mapping to be more accurate.

At the end of insertion, with the more accurate estimates of the current placement (the current estimated position/location) of the stimulating assembly within the cochlea, the neural survival map, and the selected placement (the optimal position/location), the actuator can precisely alter the position/location of the stimulating assembly to the selected placement (the optimal position/location). Thus, in example embodiments involving robotic assisted surgery, placement of the stimulating assembly can be controlled to a finer degree. Further constraints may be utilized to provide actions to the surgeon and/or the robot that are realistic (e.g., some positions of the stimulating assembly or individual electrodes thereof within the cochlea may not be physically achievable with a chosen electrode type, and may thus be excluded from consideration by the system).

### Other Example Applications of System and Techniques

In some example embodiments, the system and techniques described herein can be coupled with electrocochleography (ECochG) technology, which is used to assess cochlea hair cell survival, rather than neural cell survival. Mapping at the time of surgery provides information about neural potential, but may not necessarily correlate with neural survival post-implantation. Since measurements are taken as an electrode of the stimulating assembly passes through a region, some insertional trauma may not be accounted for in the system and methods described above. Accordingly, ECochG technology could be coupled with the system and methods described herein to provide more real-time information on electrode events that cause changes to potential neural survival.

In some example embodiments, the system and techniques described herein can be applied at the time of first fitting to generate a map that indicates “comfort levels” (C-levels) and “threshold levels” (T-levels). In certain embodiments, the magnitude of the neural survival is inverse correlated with the degree of the electrical stimulation required at a given sound presentation level. For a given electrode, if the neural survival is higher, a lesser degree of electrical stimulation is required which entails that the electrode has comparatively a lower comfort level (or “C-level” and a lower threshold level (or “T-level”). If the neural survival is lesser for a given electrode, a greater degree of electrical stimulation is required, and thus the C-level and the T-level would be comparatively higher for that electrode. The techniques described above may further include generating a map of T-levels and C-levels for the electrode array. In some example embodiments, a transformation algorithm relies upon the inverse correlation to derive an initial estimation of C-levels. The neural survival metrics are retrieved for each electrode based on positional collocation in the mathematical cochlear model. The transformation algorithm first derives the C-levels based on inverting the magnitude of the neural survival. The sets of C-levels are rescaled in range and magnitude based on normative magnitudes for first fitting. The T-levels are derived from the C-levels by subtracting the normative magnitude differences. The map of T-levels and C-levels for the electrode array are transmitted to patient's clinic for the first fitting. The clinician can then use the map to make adjustments to the T-levels and the C-levels to better suit the patient at the time of first fitting post-implantation of the electrode array.

### Summary and Illustrative Advantages

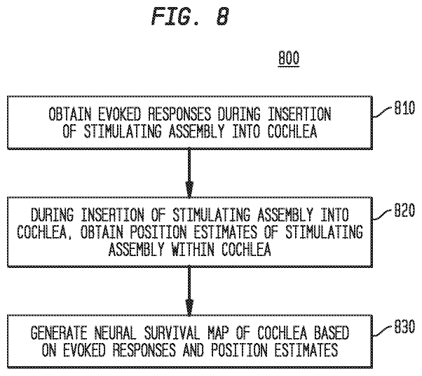

Thus, according to certain example embodiments described above and with reference to FIGS. 2, 3, 4, 5 (and also described below with reference to FIG. 8), the present invention provides a new system and techniques to utilize neural response measurements that are captured throughout surgical implantation of a stimulating assembly into the cochlea of a recipient to estimate a degree of neural survival, in further combination with electrode position estimates, to aid the collocation of the placement of the stimulating assembly (e.g., the electrodes of the electrode array) with regions of high neural survival in the cochlea through optimization of electrode position/location within the cochlea based on the neural response measurements. The techniques involve the registration of neural activity measurements (e.g., NRT, etc.) to an intra-cochlear location during insertion. The techniques also involve prompting a surgeon (or controlling a surgical robot) to adjust the position of the stimulation assembly to achieve better alignment with areas of heterogeneous neural survival. In some example embodiments, the intra-cochlear location can be obtained by impedance-based measurements (although other measures may be used as well). This disclosure covers a range of inputs which can be used to provide the neural survival map and prompt optimization of the insertion of the stimulating assembly and corresponding position/location of individual electrodes thereof.

### Example Use Cases and Applications

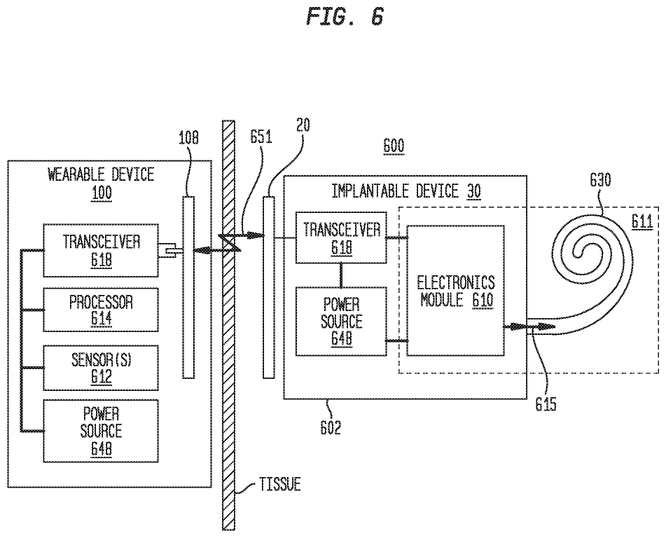

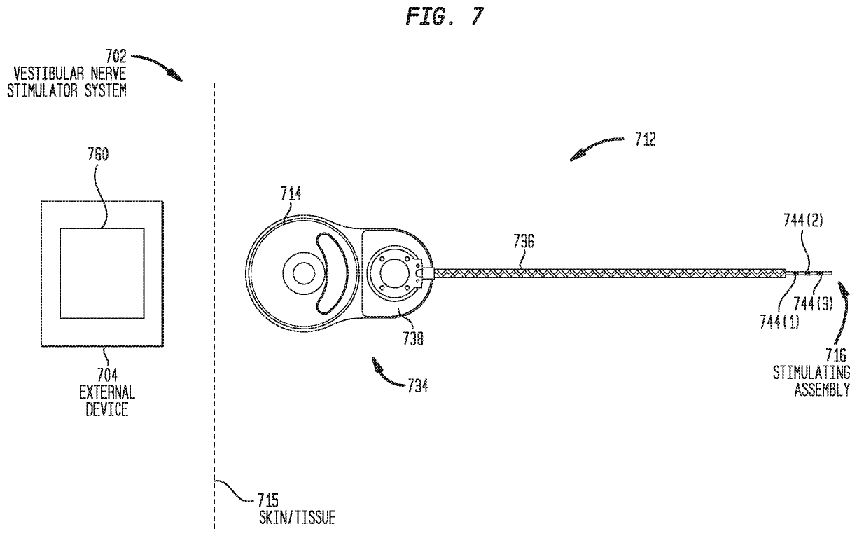

As previously described, the technology disclosed herein can be applied in any of a variety of circumstances and with a variety of different medical devices. Example medical devices that can benefit from technology disclosed herein are described in more detail in FIGS. 6 and 7 below. As described below, the operating parameters for the devices described with reference to FIGS. 6 and 7 can be configured according to the techniques described herein. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue, to the extent that the operating parameters of such devices can be tailored based upon the posture of the user receiving the device. Further, technology described herein can also be applied to consumer devices. These different systems and devices can benefit from the technology described herein. For example, the operation techniques of the present disclosure can be applied to consumer grade or commercial grade headphone or ear bud products.

FIG. 6 is a functional block diagram of an implantable stimulator system 600 that can benefit from the technologies described herein. The implantable stimulator system 600 includes a wearable device 100 acting as an external processor device, and an implantable device 30 acting as an implanted stimulator device. In examples, the implantable device 30 is an implantable stimulator device configured to be implanted beneath a user's tissue (e.g., skin). In examples, the implantable device 30 includes a biocompatible implantable housing 602. Here, the wearable device 100 is configured to transcutaneously couple with the implantable device 30 via a wireless connection to provide additional functionality to the implantable device 30.

In the illustrated example, the wearable device 100 includes one or more sensors 612, a processor 614, a transceiver 618, and a power source 648. The one or more sensors 612 can be one or more units configured to produce data based on sensed activities. In an example where the stimulation system 600 is an auditory prosthesis system, the one or more sensors 612 include sound input sensors, such as a microphone, an electrical input for a frequency modulation (FM) hearing system, other components for receiving sound input, or combinations thereof. Where the stimulation system 600 is a visual prosthesis system, the one or more sensors 612 can include one or more cameras or other visual sensors. Where the stimulation system 600 is a cardiac stimulator, the one or more sensors 612 can include cardiac monitors. The processor 614 can be a component (e.g., a central processing unit) configured to control stimulation provided by the implantable device 30. The stimulation can be controlled based on data from the one or more sensors 612, a stimulation schedule, or other data. Where the stimulation system 600 is an auditory prosthesis, the processor 614 can be configured to convert sound signals received from the sensor(s) 612 (e.g., acting as a sound input unit) into signals 651. The transceiver 618 is configured to send the signals 651 in the form of power signals, data signals, combinations thereof (e.g., by interleaving the signals), or other signals. The transceiver 618 can also be configured to receive power or data. Stimulation signals can be generated by the processor 614 and transmitted, using the transceiver 618, to the implantable device 30 for use in providing stimulation.

In the illustrated example, the implantable device 30 includes a transceiver 618, a power source 648, and a medical instrument 611 that includes an electronics module 610 and a stimulation assembly 630. The implantable device 30 further includes a hermetically sealed, biocompatible implantable housing 602 enclosing one or more of the components.

The electronics module 610 can include one or more other components to provide medical device functionality. In many examples, the electronics module 610 includes one or more components for receiving a signal 651 and converting the signal 651 into a stimulation signal 615. The electronics module 610 can further include a stimulator unit. The electronics module 610 can generate or control delivery of the stimulation signals 615 to the stimulation assembly 630. In examples, the electronics module 610 includes one or more processors (e.g., central processing units or microcontrollers) coupled to memory components (e.g., flash memory) storing instructions that when executed cause performance of an operation. In examples, the electronics module 610 generates and monitors parameters associated with generating and delivering the stimulus (e.g., output voltage, output current, or line impedance). In examples, the electronics module 610 generates a telemetry signal (e.g., a data signal) that includes telemetry data. The electronics module 610 can send the telemetry signal to the wearable device 100 or store the telemetry signal in memory for later use or retrieval.

The stimulation assembly 630 can be a component configured to provide stimulation to target tissue. In the illustrated example, the stimulation assembly 630 is an electrode assembly that includes an array of electrode contacts disposed on a lead. The lead can be disposed proximate tissue to be stimulated. Where the system 600 is a cochlear implant system, the stimulation assembly 630 can be inserted into the user's cochlea. The stimulation assembly 630 can be configured to deliver stimulation signals 615 (e.g., electrical stimulation signals) generated by the electronics module 610 to the cochlea to cause the user to experience a hearing percept. In other examples, the stimulation assembly 630 is a vibratory actuator disposed inside or outside of a housing of the implantable device 30 and configured to generate vibrations. The vibratory actuator receives the stimulation signals 615 and, based thereon, generates a mechanical output force in the form of vibrations. The actuator can deliver the vibrations to the skull of the user in a manner that produces motion or vibration of the user's skull, thereby causing a hearing percept by activating the hair cells in the user's cochlea via cochlea fluid motion.

The transceivers 618 can be components configured to transcutaneously receive and/or transmit a signal 651 (e.g., a power signal and/or a data signal). The transceiver 618 can be a collection of one or more components that form part of a transcutaneous energy or data transfer system to transfer the signal 651 between the wearable device 100 and the implantable device 30. Various types of signal transfer, such as electromagnetic, capacitive, and inductive transfer, can be used to usably receive or transmit the signal 651. The transceiver 618 can include or be electrically connected to a coil 20.

As illustrated, the wearable device 100 includes a coil 108 for transcutaneous transfer of signals with the coil 20. As noted above, the transcutaneous transfer of signals between the coil 108 and the coil 20 can include the transfer of power and/or data from the coil 108 to the coil 20 and/or the transfer of data from the coil 20 to the coil 108. The power source 648 can be one or more components configured to provide operational power to other components. The power source 648 can be or include one or more rechargeable batteries. Power for the batteries can be received from a source and stored in the battery. The power can then be distributed to the other components as needed for operation.

As should be appreciated, while particular components are described in conjunction with FIG. 6, technology disclosed herein can be applied in any of a variety of circumstances. The above discussion is not meant to suggest that the disclosed techniques are only suitable for implementation within systems akin to that illustrated in and described with respect to FIG. 6. In general, additional configurations can be used to practice the methods and systems herein and/or some aspects described can be excluded without departing from the methods and systems disclosed herein.

FIG. 7 illustrates an example vestibular nerve stimulator system 702, with which embodiments presented herein can be implemented. As shown, the vestibular nerve stimulator system 702 comprises an implantable component (vestibular stimulator) 712 and an external device/component 704 (e.g., external processing device, battery charger, remote control, etc.). The external device 704 comprises a transceiver unit 760. As such, the external device 704 is configured to transfer data (and potentially power) to the vestibular stimulator 712. External device 704 can also include an inertial measurement unit analogous to inertial measurement unit 170 of FIG. 1D.

The vestibular stimulator 712 comprises an implant body (main module) 734, a lead region 736, and a stimulating assembly 716, all configured to be implanted under the skin (tissue) 715 of the user. The implant body 734 generally comprises a hermetically-sealed housing 738 in which RF interface circuitry, one or more rechargeable batteries, one or more processors, and a stimulator unit are disposed. The implant body 134 also includes an internal/implantable coil 714 that is generally external to the housing 738, but which is connected to the transceiver via a hermetic feedthrough (not shown). Implant body 734 can also include an inertial measurement unit analogous to inertial measurement unit 180 of FIG. 1D.

The stimulating assembly 716 comprises a plurality of electrodes 744(1)-(3) disposed in a carrier member (e.g., a flexible silicone body). In this specific example, the stimulating assembly 716 comprises three (3) stimulation electrodes, referred to as stimulation electrodes 744(1), 744(2), and 744(3). The stimulation electrodes 744(1), 744(2), and 744(3) function as an electrical interface for delivery of electrical stimulation signals to the user's vestibular system.

The stimulating assembly 716 is configured such that a surgeon can implant the stimulating assembly adjacent the user's otolith organs via, for example, the user's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 712, the external device 704, and/or another external device, can be configured to implement the techniques presented herein. That is, the vestibular stimulator 712, possibly in combination with the external device 704 and/or another external device, can include an evoked biological response analysis system, as described elsewhere herein.

FIG. 8 is a flowchart of an example method 800 for generating a neural survival map of a cochlea, which may be implemented using the system described herein in accordance with certain embodiments presented herein. As shown in FIG. 8, after the flow commences, the system obtains a plurality of evoked responses during insertion of the stimulating assembly into the cochlea at operation 810. In some example embodiments, operation 810 includes iteratively delivering electrical stimulation signals to the cochlea, and capturing electrically evoked compound action potentials (ECAPs) in response to each iteration of electrical stimulation signals delivered to the cochlea.

Further, during insertion of the stimulating assembly into the cochlea, the system obtains position estimates of the stimulating assembly within the cochlea at operation 820. For example, operation 820 may include iteratively estimating locations of the stimulating assembly relative to a multi-dimensional geometric model of the cochlea. In some examples, operation 820 may include capturing a plurality of impedance measurements (e.g., two-point impedance measurements, two-point transimpedance measurements, or a combination thereof), and determining the position estimates based at least in part on the impedance measurements. In other examples, operation 820 may include capturing a plurality of accelerometer measurements, and determining the position estimates based at least in part on the accelerometer measurements.

At operation 830, the system then generates a neural survival map of the cochlea based on the evoked responses and the position estimates. In some example embodiments, each of the plurality of evoked responses is registered to (collocated with) one of the plurality of position estimates to generate the neural survival map.

In some example embodiments, the neural survival map may be utilized to determine a selected placement (an optimal position/location) for the stimulating assembly within the cochlea, and determine a positional adjustment to the stimulating assembly for achieving the selected placement based on a current estimated position of the stimulating assembly.

In some example embodiments, the positional adjustment may be output (e.g., displayed to a surgeon on a display device, or transmitted to control a surgical robot) to allow for manual or automatic positional adjustment of the stimulating assembly that achieves the selected placement (optimal position/location) of the stimulating assembly within the cochlea.

### Additional Variations and Alternatives

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

What is claimed is:

1. A method comprising:
during insertion of a stimulating assembly into a cochlea body cavity, obtaining a plurality of evoked responses from within the body cavity;
during insertion of the stimulating assembly into the body cavity, obtaining a plurality of position estimates of the stimulating assembly within the body cavity; and
generating a neural map of the body cavity based on the plurality of evoked responses and the plurality of position estimates.

2. The method of claim 1, wherein generating the neural map of the body cavity based on the plurality of evoked responses and the plurality of position estimates comprises:
collocating each of the plurality of evoked responses with one of the plurality of position estimates.

3. The method of claim 1, further comprising:
generating a multi-dimensional geometric model of the body cavity, wherein obtaining the plurality of position estimates of the stimulating assembly within the body cavity comprises:
iteratively estimating locations of the stimulating assembly relative to the multi-dimensional geometric model.

4. The method of claim 1, wherein obtaining the plurality of evoked responses during insertion of the stimulating assembly into the body cavity comprises:
iteratively delivering electrical stimulation signals within the body cavity; and
capturing electrically evoked compound action potentials (ECAPs) in response to each iteration of electrical stimulation signals delivered within the body cavity.

5. The method of claim 1, wherein obtaining the plurality of position estimates of the stimulating assembly within the body cavity comprises:
capturing a plurality of impedance measurements; and
determining the plurality of position estimates at least partially based on the plurality of impedance measurements.

6. (canceled)

7. (canceled)

8. The method of claim 1, wherein obtaining the plurality of position estimates of the stimulating assembly within the body cavity comprises:
capturing a plurality of accelerometer measurements; and
determining the plurality of position estimates at least partially based on the plurality of accelerometer measurements.

9. (canceled)

10. The method of claim 3, further comprising:
retrieving at least one of a pre-operative medical imaging scan data or user input data describing dimensions of the body cavity, wherein the dimensions include length, width, and height of the body cavity; and
generating the multi-dimensional geometric model of the body cavity based on the at least one of the pre-operative medical imaging scan data or the user input data describing the dimensions of the body cavity.

11. The method of claim 1, further comprising:
mapping an internal structure of the body cavity while obtaining a plurality of evoked responses from within the body cavity.

12. (canceled)

13. (canceled)

14. The method of claim 1, further comprising:
determining whether the stimulating assembly is still being inserted into the body cavity or insertion has ceased.

15. The method of claim 1, further comprising:
determining, based on the neural map, a selected placement for the stimulating assembly within the body cavity; and
determining, based on a current estimated position of the stimulating assembly within the body cavity, a positional adjustment to the stimulating assembly for achieving the selected placement for the stimulating assembly within the body cavity.

16. The method of claim 15, wherein the stimulating assembly comprises a plurality of electrodes, and wherein determining the selected placement for the stimulating assembly comprises:
determining a placement for the stimulating assembly that maximizes alignment of the plurality of electrodes with populations of surviving nerve cells based on the neural map.

17. The method of claim 15, wherein determining the positional adjustment to the stimulating assembly comprises:
comparing a current estimated position of the stimulating assembly within the body cavity with the selected placement for the stimulating assembly; and
determining a direction and a magnitude for the positional adjustment based on the comparing.

18. The method of claim 15, further comprising:
generating an output representing at least the positional adjustment to the stimulating assembly for achieving the selected placement for the stimulating assembly.

19. The method of claim 15, further comprising:
estimating comfort levels for electrodes of the stimulating assembly based on positional collocation of the electrodes with corresponding intra-operative neural response measurements in a multi-dimensional geometric model of the body cavity;
deriving threshold levels (T-levels) the electrodes of the stimulating assembly based on the comfort levels; and
generating a map of the comfort levels and the threshold levels for the electrodes of the stimulating assembly.

20-32. (canceled)

33. The method of claim 15, further comprising:
generating an output to control a robotic surgical device to adjust positioning of the stimulating assembly within the body cavity based on the positional adjustment to the stimulating assembly.

34. The method of claim 15, further comprising:
determining whether the selected placement for the stimulating assembly has been achieved.

35. One or more non-transitory computer readable storage media comprising instructions that, when executed by a processor, cause the processor to:
obtain a plurality of evoked responses during insertion of a stimulating assembly into a body cavity of a recipient;
during insertion of the stimulating assembly into the body cavity of the recipient, obtain a plurality of position estimates of the stimulating assembly within the body cavity; and
generate a neural map of the body cavity based on the plurality of evoked responses and the plurality of position estimates.

36. The one or more non-transitory computer readable storage media of claim 35, wherein the body cavity is an inner ear of the recipient.

37. (canceled)

38. The one or more non-transitory computer readable storage media of claim 35, further comprising instructions operable to:
collocate each of the plurality of evoked responses with one of the plurality of position estimates.

39. The one or more non-transitory computer readable storage media of claim 35, further comprising instructions operable to:
determine, based on the neural map, a selected placement for the stimulating assembly within the body cavity; and
determine, based on a current estimated position of the stimulating assembly within the body cavity, a positional adjustment to the stimulating assembly for achieving the selected placement for the stimulating assembly within the body cavity.

40-45. (canceled)
